(19) United States
(12) Patent Application Publication
(10) Pub. No.: US 2007/0179560 A1
(43) Pub. Date: Aug. 2, 2007

(54) Functional electrical stimulation system

(76) Inventors: Kai-Yu Tong, Hong Kong (CN); Arthur F. T. Mak, Hong Kong (CN); Kelvin T. Y. Leung, Hong Kong (CN)

Correspondence Address: Thomas E. Sisson;Jackson Walker, LLP, Suite 2400, 112 E. Pecan, San Antonio, TX, 78205, US

(21) Appl. No.: 11/650,778

(22) Filed: Jan. 8, 2007

Related U.S. Application Data
(63) Continuation-in-part of application No. 10/278,575, filed on Oct. 23, 2002, now Pat. No. 7,162,305.

(51) Int. Cl. A61N 1/00

(52) U.S. Cl. 607/48

(57) ABSTRACT

An electrical stimulation device for a body part of a person comprises an orthosis with sensor and electrodes and a controller. The controller receives a sensor signal, compares the sensor signal to a threshold value and generates an electrical output from the electrodes if the sensor signal exceeds the threshold value. The controller comprises a calibration module for determining an initial resting position of the body part.

This is a continuation-in-part of co-pending U.S. patent application Ser. No. 10/278,575, filed Oct. 23, 2002.

### Background to the Invention

The present invention relates to Functional Electrical Stimulation Systems. Functional Electrical Stimulation (FES) systems artificially stimulate the muscles, and muscle groups, of persons through the use of electrical current in order to stimulate movement. As early as 1971 Liberson applied electrical stimulation to assist walking in patients with foot drop. Current FES systems are mainly designed for persons after a spinal cord injury and stroke.

Several different groups of researchers have designed implant electrodes and systems for spinal cord injured persons. These systems need to be surgically implanted into muscle groups and are aimed at control of deep muscles.

After stroke patients often suffer from weakness in the extensor muscle on the upper limb and involuntary muscular contractions due to spasticity of the flexor muscle groups. FES can be used with stroke victims to stimulate the extensor muscle to open the hand and reduce the spasticity. The FES can be used for therapeutic training and some patients have been found to partially or completely recover hand functions eliminating the need for an implant system.

### Brief Description of the Drawings

Embodiments of the invention will now be described with reference to the accompanying drawings in which:

FIG. 1 is a schematic of a first configuration of a Functional Electrical Stimulation (FES) system according to the invention,

FIG. 2 is a schematic of a second configuration of the Functional Electrical Stimulation (FES) system,

FIG. 3 is a perspective view of an Orthosis of the FES system,

FIG. 4 is a side view of the orthosis,

FIG. 5 is a bottom view of the orthosis,

FIG. 6 is a second perspective view of the orthosis,

FIG. 7 is an illustration of a Portable Stimulation Unit for the FES system,

FIG. 8 is a schematic view of the control system for the Portable Stimulation Unit,

FIG. 9 is an illustration of a computer graphical user interface for use with the FES system,

FIG. 10 is a flow diagram of a control strategy of the FES system,

FIG. 11 is a perspective view of a second embodiment of an Orthosis of the FES system, and

FIG. 12 is a graphic illustration of triggering values for stimulation from the sensor of the second embodiment.

### Description of the Preferred Embodiments

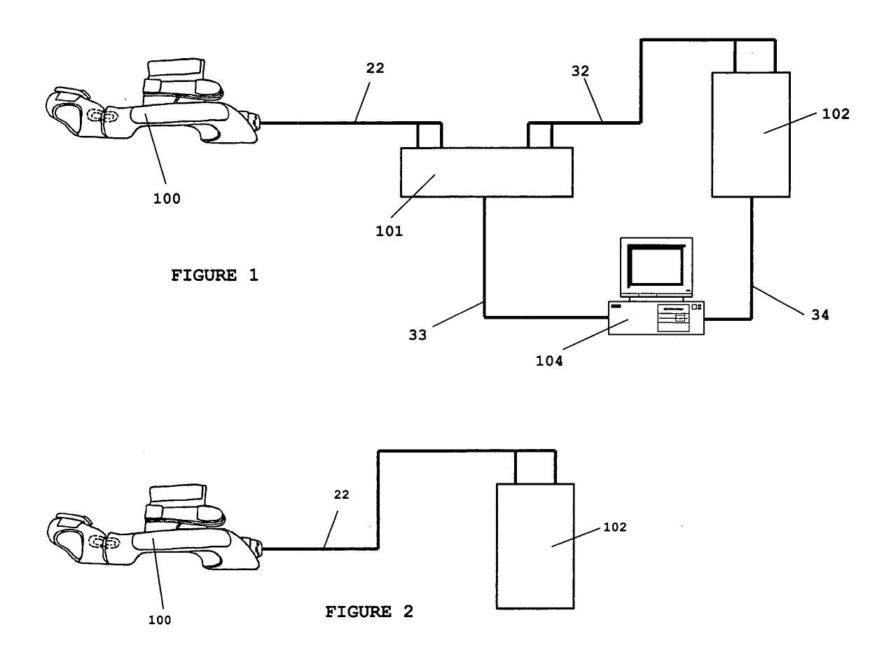

FIGS. 1 and 2 are schematics of a Functional Electrical Stimulation (FES) system. It consists of three basic parts: a hand-wrist orthosis 100, a portable stimulation unit 102 and a docking station 101. The FES interfaces with a computer 104. The functional electrical stimulation system has two basic configurations. FIG. 1 illustrates the first configuration, which is for training and parameter setup. FIG. 2 illustrates the second configuration in which the portable stimulation unit works in standalone mode without connection to the docking station. These configurations will be described in more detail later.

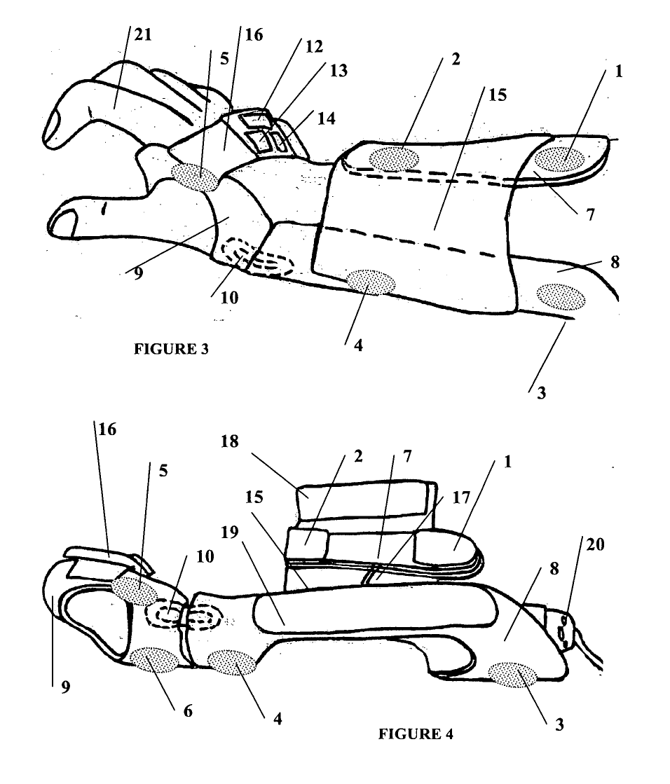

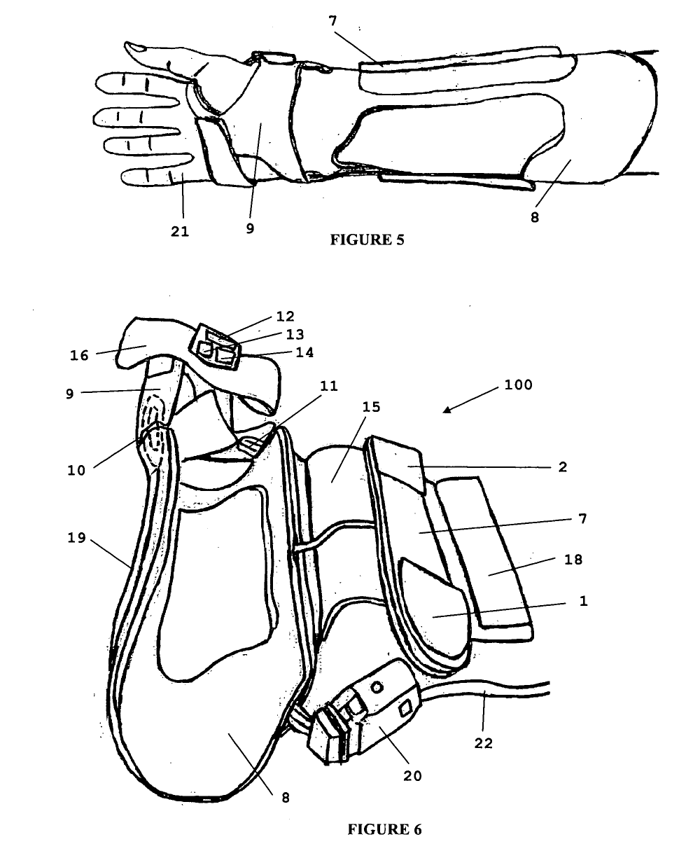

Referring to FIGS. 3 to 6, the hand-wrist orthosis 100 is made of plastic material moulded to fit the shape of the forearm of a stroke affected hand 21 of a patient. The orthosis 100 comprises three pieces. These are: a posterior portion 7, anterior portion 8, and a hand portion 9. The hand portion 9 is connected with the anterior portion 8 by two joints 10, 11 on the lateral sides. The joints 10, 11 allow flexibility for wrist movement. A strap 15 is attached to one side of the anterior portion 8 and passes over the top part of the patients forearm to detachably connect with the other side of the anterior portion 8 by Velcro™ fasteners 18, 19. The posterior aspect 7 is mounted on the strap 15 for location on the top part of the patients forearm.

The hand portion 9 includes two electrodes 5, 6. A Thenar electrode 5 is for stimulating the Thenar muscle group and a thumb electrode 6 is for stimulating the thumb abductor. The posterior portion 7 includes two electrodes 1, 2 for stimulating the wrist extensor muscle group. The anterior portion 8 includes two electrodes 3 and 4 for stimulating the wrist flexor muscle group. The electrodes 1, 2, 3, 4, 5, 6 are self-adhesive type electrodes located on the inner surface of the orthosis 100 to correspond to the above mentioned muscle groups. The electrodes are located by a clinician to suit the patient.

For stimulating the Thenar group electrode 5 is the Active pole and electrode 4 is the Indifferent pole. For stimulating the Thumb abductor electrode 6 is the Active pole and electrode 4 is the Indifferent pole. For stimulating the Wrist extensor electrode 1 is the Active pole and electrode 2 is the Indifferent pole. And for stimulating the Wrist flexor electrode 3 is the Active pole and electrode 4 is the Indifferent pole. An Active pole is the negative terminal and an Indifferent pole is the positive terminal.

A pressure sensor 12, accelerometer 13 and gyroscope 14 are located on a strap 16 on the back of hand portion 9. The sensors provide feedback of hand movement and position.

Signal wires from sensors and electrodes on the orthosis 100 are brought together at a connector 20 on the anterior portion 8. The orthosis 100 is linked to the portable stimulation unit 102 or docking station 101 by signal cable 22.

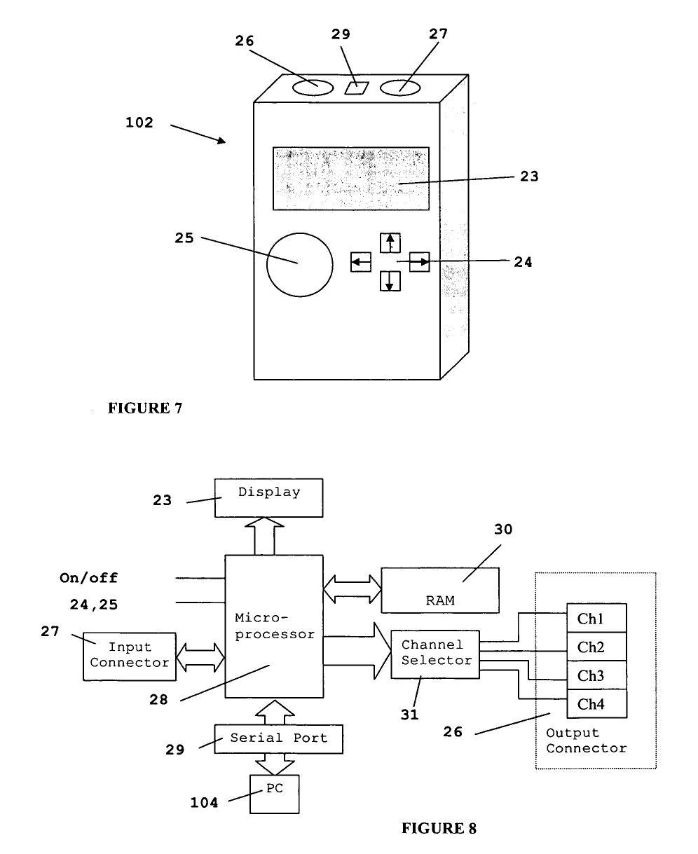

Referring to FIGS. 7 and 8, the portable stimulation unit 102 generates a train of electrical pulse, which it transmits to selected electrodes to stimulate selected muscles and coordinate muscle contractions. The portable simulation unit 102 is controlled by a microprocessor 28. Simulation parameters are stored in Random Access Memory (RAM) 30. An output channel selector 31 and electrode output connector 26 transmit the train of electrical pulse to the electrodes. Feedback from the sensors is input to the microprocessor 28 via input connector 27.

Manual control of the portable simulation unit 102 is provided by a user interface means comprising a 12×2 Liquid Crystal Display 23, up/down/left/right input buttons 24 and a selection input button 25 on a front panel of the portable stimulation unit 102. The LCD display 25 provides information about the stimulation patterns and the user can adjust parameters such as the stimulation frequency, stimulation amplitude levels, sensor-threshold values, pulse widths, duration time using the interface means.

The portable stimulation unit B can interface with the computer 104 via a serial port 29 to facilitate download of simulation parameters obtained during setup and training.

The docking station 101 comprises two microprocessors and input and output connectors for the sensors and electrodes. Both the input and output connectors are connected to an input microprocessor for capture of real-time signals from the sensors and feedback of electrode output signal parameters. The output connector is connected to an output microprocessor for output of electrode control signals. The docking station 101 also includes a parallel connector for interface to the host computer 104 that facilitates graphical displays showing input and output signal parameters, parameter adjustment and data logging.

The functional electrical stimulation system has two basic configurations. A first configuration is for training and parameter setup. In this first configuration the connector lead 22 from sensor and electrodes on the orthosis 100 are connected to the input/output connectors of the docking station 101, and an extension cable 32 from the docking station 101 connect to the portable simulation unit 102. The docking station 101 also includes a parallel connector 33 for interface to the host computer 104 that facilitates graphical displays showing input and output signal parameters, parameter adjustment and data logging. At the end of the training and parameter setup session the simulation parameters are uploaded from the computer 104 to the Portable Simulation Unit 102 via a serial connection 34.

In a second configuration the orthosis 100 and portable stimulation unit 102 work in standalone mode without connection to the docking station 101 or computer 104. In this second configuration the input/output cables 22 from the orthosis 100 connect directly to the portable stimulation unit 102. This allows the patient to go home, or go about there daily routine, without the need to carry/wear bulky equipment. The portable simulation unit 102 responds to input signals to generate output signals according to the simulation parameters uploaded from the computer 104. Minor adjustment of simulation parameters and control of the portable simulation unit 102 are achieved via the user interface means.

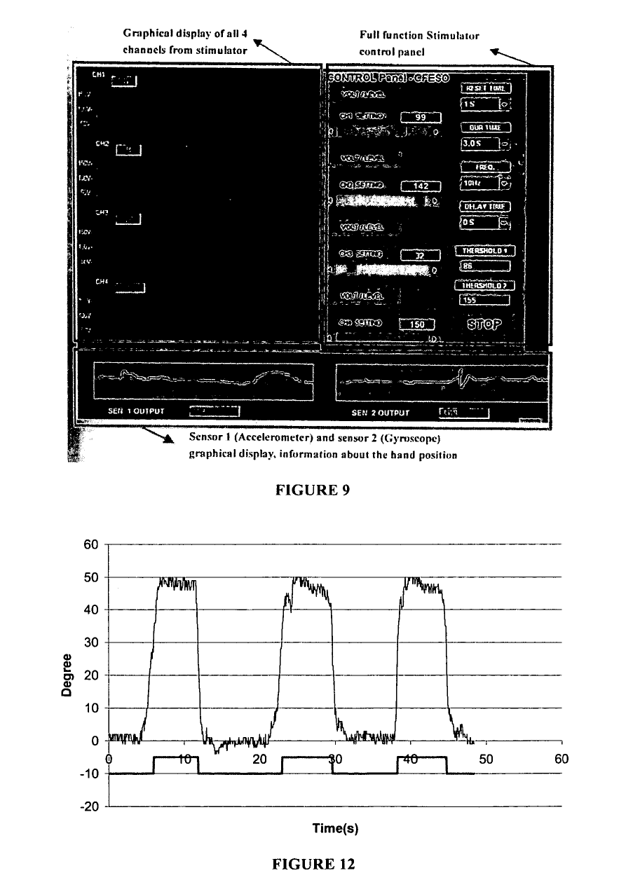

Referring to FIG. 9, a layout of a Graphical User Interface on a computer is shown. A Microsoft Windows based Graphical User Interface can be programmed in Visual Basic or constructed using an application such as ‘labVIEW’ available from National Instruments (www.ni.com).

The simulation parameters and their respective range are set out in the following table.

| | |
|---|---|
| Input 1 Threshold 1 | 256 levels; 0 to 5 Volts |
| Input 2 Threshold 2 | 256 levels; 0 to 5 Volts |
| Output Channel 1 Amplitude | 256 levels; 0 to 100 mA |
| Output Channel 2 Amplitude | 256 levels; 0 to 100 mA |
| Output Channel 3 Amplitude | 256 levels; 0 to 100 mA |
| Output Channel 4 Amplitude | 256 levels; 0 to 100 mA |
| Output Frequency | 10 Hz, 20 Hz, 30 Hz, 40 Hz, or 50 Hz |
| Output Pulse Width | 256 levels: 0-500 □s |
| Reset Time | 0 to 9 Seconds |
| Output Delay time | 0 to 4.5 Seconds |
| Output Duration Time | 0 to 99 Seconds |

The microprocessor may set the output on each channel to one of 256 levels within a range of 1 to 100 mA. The required level for each channel, to achieve the required muscle group stimulation, is determined during the setup mode (configuration 1) where monitoring feedback of the output parameters is possible. This level is stored in the portable stimulation unit for recall during standalone use. The muscle groups stimulated by each channel are:

| | |
|---|---|
| Channel 1: | Electrode 6 and 4 for stimulating the thumb abductor. |
| Channel 2: | Electrode 5 and 4 for stimulating the Thenar muscle group. |
| Channel 3: | Electrodes 1 and 2 for stimulating the wrist extensor |
| | muscle group. |
| Channel 4: | Electrodes 3 and 4 for stimulating the wrist flexor muscle |
| | group. |

The sensors are 0 to 5 volts with a resolution of 256. The two input thresholds are set a one of 256 levels.

The input channels are associated with the following sensors:

| | |
|---|---|
| channel 1: | The Accelerometer for detecting the tilt angle of the palm |
| | during wrist extension. |
| Channel 2: | The gyroscope for detecting the lateral rotation of the wrist by |
| | measuring the angular velocity. |

A pressure sensor is also included on the orthosis. It provides an on/off switch to trigger the Portable Stimulator Unit for mode 2 stimulation (described below). It is a very thin for the pressure sensor, therefore the sensor can be attached on the surface of the sensor cluster.

The functional electrical stimulation system has 3 control modes selectable at the portable simulation unit.

Mode 1 is a simple exercise control. The patient chooses a muscle group to exercise and the simulation unit repeatedly stimulates that muscle group until the patient exits the mode.

Mode 2 is a manual mode in which the patient manually initiates a single stimulation of a selected muscle group. The pressure sensor on the orthosis can be used to initiate the stimulation.

Mode 3 is an automated stimulation mode in which the portable stimulation unit monitors feedback from the sensors and initiates a stimulation if the inputs exceed the input thresholds. The sensors capture the patient's intention from their voluntary residual movement on the affected upper limb. This mode can initiate two different types of movement and then generate two different stimulation patterns controlling two different hand postures: hand opening for spastic hand, and Lateral Grasp for holding a pen.

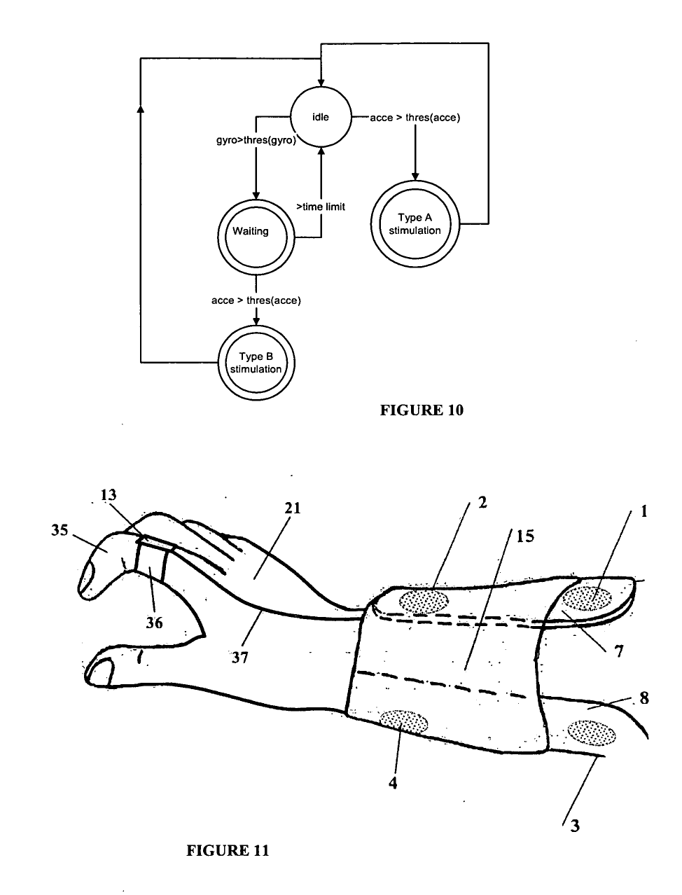

Referring to FIG. 10, if the accelerometer sensor signal on input channel 1 is above threshold 1 the system enters stimulation A control. The microprocessor waits for the period set in the “Delay Time” parameter and then activates both Channel 1 (Electrodes 6) and Channel 3 (Electrodes 1 and 2) output signals to stimulate the respective muscles/muscle groups. The channels are closed again after the period of time set in the “Duration Time” parameter. The microprocessor enters an Idle stage for the period of time set in the “Reset Time” parameter. This is to prevent the functional electrical stimulation system immediately repeating the stimulation if the input 1 signal is still above the threshold 1 value.

A stimulation B is triggered by lateral wrist rotation followed by wrist extension. If the gyroscope sensor input on channel 2 is above the input 2 threshold the microprocessor will enter the Waiting stage. If the accelerometer sensor signal on input channel 1 does not go above threshold 1 within the “Reset time”, then the microprocessor will return back to Idle stage. If the accelerometer sensor signal on input channel 1 goes above threshold 1 within the “Reset Time” the microprocessor enters Stimulation B control. The microprocessor waits for the period set in the “Delay Time” parameter and then activates both Channel 2 (Electrodes 5) and Channel 3 (Electrodes 1 and 2). The channels are closed again after the period of time set in the “Duration Time” parameter. The microprocessor enters an Idle stage for the period of time set in the “Reset Time” parameter. This is to prevent the functional electrical stimulation system immediately repeating the stimulation if the input signals is still above the threshold values.

The functional electrical stimulation system triggers the stimulation pattern through the sensors to capture the patient's intention from their voluntary residual movement on the affected upper limb. There are lots of patients after stoke who still have partially voluntary movement on hand and wrist. By encouraging their hand movement, the patients can gradually re-learn the function movement. The present invention could help the user to motor-relearn the functional movements.

Although the orthosis of FIGS. 3-6 is easily fitted by a clinician some stroke patients can have difficulty putting on an orthosis with hand portion 9. Additionally, some stroke patients might only be able to move one or two fingers. Therefore, in a second embodiment of the invention the hand portion 9 is replaced with a finger sensor that can detect both finger and wrist motion at the same time. The finger sensor is designed as a ring with an elastic band 36 and an accelerometer 13 which can be more easily fitted by the patient themselves. The accelerometer 13 is a dual-axis accelerometer (for example an ADXL311 by Analog Device, USA) that measures gravitational forces and is used as a tilt sensor to detect finger and wrist extension and rotation. If a person can move their finger or extend their wrist after a stroke, then the accelerometer 13 is used to detect the changes in the inclination on the finger with the horizontal plane. This signal is used to trigger the stimulation.

However, as the sensor is on the distal end on the limb it experiences a large degree of freedom of movement and the forearm movement due to gravity affects the success rate in controlling the FES system. To overcome these problems a calibration function is used to determine the initial resting position of the distal end of upper limb prior to use of the FES system. Upon fitting or putting on the orthosis the patient or clinician activates the auto-calibration function for the FES system to determine the resting position of the limb. Auto-calibration samples the accelerometer 13 signal for 1 second at a sampling rate of 20 Hz. The averaged value of the samples is used to calculate the initial resting angle of the limb using the equation Resting Angle θ=Arcsine[(Average Sensor Signal)/Gravity].

Once auto-calibration is complete the ES system automatically enters stimulation mode where stimulation is triggered when the tilt angle of the hand exceeded a threshold angle from the calculated resting angle θ. The threshold angle is determined for each patient and can be calculated by determining the maximum range of residual movement, for example wrist extension and or rotation, of the patient. FIG. 12 graphically illustrates the sensor signal, top plot, and the corresponding electrical stimulation output, bottom plot, when the sensor signal exceeds the threshold value (for example 30 degree). In accordance with the first embodiment the controller can stimulate different muscle groups in response to either or both wrist extension and or rotation.

If the patient changes their resting position during use of the FES system they can re-activate the auto-calibration so that the FES system automatically determines the new resting position. It is envisaged however, that the FES system may automatically re-calibrate the resting position automatically without patient or clinician input. For example, the FES system could automatically re-calibrate if it detects that there has been no movement of the limb (hand) for a certain length of time and the resting angle has been changed from previous resting position, or immediately after stimulation when the limb is moved to a new position, or if the limb is not moved for a certain length of time after stimulation.

Where in the foregoing description reference has been made to integers or elements having known equivalents then such are included as if individually set forth herein.

Embodiments of the invention have been described, however it is understood that variations, improvements or modifications can take place without departure from the spirit of the invention or scope of the appended claims.

What is claimed is:

1. An electrical stimulation device for aiding movement of a partially paralysed body part, comprising:
an orthosis for wearing on a body part of a person,
a sensor on the orthosis for detecting movement of the body part and if movement of the body part is detected producing a sensor signal,
an electrode on the orthosis for contacting the skin surface over a muscle used for movement of the body part, and
a controller in electrical communication with the sensor and electrode, the controller having a stimulation module for receiving the sensor signal, comparing the sensor signal to a threshold value and if the sensor signal exceeds the threshold value generating an electrical output from the electrode to the muscle so as to stimulate movement of the body part in a way indicate by the movement.

2. The electrical stimulation device of claim 1 wherein the body part is the hand and forearm of the person, the sensor detecting residual unaided movement of the hand and forearm and the electrode contacting the skin surface over muscles of the hand and/or forearm for stimulating movement of the hand and forearm in a way indicate by the residual unaided movement.

3. The electrical stimulation device of claim 2 wherein residual unaided movement is extension or rotation of the wrist.

4. The electrical stimulation device of claim 2 wherein the muscles of the hand and/or forearm are thumb abductor, thenar, wrist extensor, and wrist flexor muscles.

5. The electrical stimulation device of claim 1 or claim 2 wherein the orthosis includes a first part for wearing on the forearm of the person and a second part for wearing on a finger of the person, and wherein the sensor is on the second part and the electrode is on the first part.

6. The electrical stimulation device of claim 5 wherein the sensor is a dual axial accelerometer.

7. The electrical stimulation device of claim 5 wherein the controller comprises a calibration module for determining an initial resting position of the body part whenever the controller is activated.
